(19) United States
(12) Patent Application Publication
(10) Pub. No.: US 2005/0124869 A1
(43) Pub. Date: Jun. 9, 2005

(54) Non-invasive, in vivo substance measurement systems

(76) Inventors: John Hefti, San Francisco, CA (US); Joseph Page, Valbonne (FR); James Plante, San Diego, CA (US)

Correspondence Address: Joseph Page, P.O. Box 757, La Jolla, CA, 92038, US

(21) Appl. No.: 10/728,992

(22) Filed: Dec. 8, 2003

(51) Int. Cl. A61B 5/00

(52) U.S. Cl. 600/316; 600/310

(57) ABSTRACT

Tissue substance measurement systems are arranged with highly specialized optical sources. Quantum cascade lasers are high power semiconductor lasers which are tiny in size and highly tunable with respect to wavelength. When deployed in non-invasive tissue substance measurement systems, quantum cascade lasers offer system advantages such as high accuracy, small size, convenience, efficiency, among others. These specialized semiconductors may be used with systems based upon photoacoustic principles. Systems may be formed of a plurality of quantum cascade laser in an optical source, mechanism to couple light into tissue, an acoustic detector and a signal processor. In some versions, user interfaces provide a reporting and feedback function to a user.

### BACKGROUND OF THE INVENTIONS

Field

The following inventions disclosure is generally concerned with in-vivo measurement of human blood for analyte concentrations. More particularly, these inventions concern systems for advanced detection of blood analyte levels including glucose concentration via specialized optical illumination strategies.

The American Diabetes Association ADA reports that presently over 6% of Americans, more than 17 million people, have diabetes. The future is grim. One in three U.S. children born in 2000 will become diabetic unless many more people start eating less and exercising more, scientists with the Centers for Disease Control CDC warn. The number of diagnosed cases of diabetes rose by nearly half in just the past 10 years, hitting 11 million in 2000, and is expected to rise by 2050- to 29 million—an earlier CDC study found.

Worldwide, the numbers are startling: the World Health Organization estimates that by 2025, the number of people with diabetes worldwide will more than double, from 140 million to 300 million.

The ADA further reports that diabetes is a leading cause of death in the United States, contributing to nearly 200,000 deaths per year. Diabetes is a chronic disease having no cure. Complications of the disease include blindness, kidney disease, nerve disease, heart disease, and stroke among others. Diabetes may be a leading cause of new cases of blindness in individuals ages between 20 and 70. Between 12,000-24,000 people per year lose their sight because of diabetes. Diabetes is the leading cause of end-stage renal disease, accounting for nearly 40% of new kidney disease cases. Nearly 60-70% of people with diabetes have mild to severe forms of diabetic nerve damage which, in severe forms, can lead to lower limb amputations. People with diabetes are 2-4 times more likely to have heart disease and to suffer strokes.

The odds are worse for black and Hispanic children: nearly half of them are likely to develop the disease, according to Dr. K. M. Venkat Narayan, a diabetes epidemiologist at the CDC.

Diabetes is a disease in which the body does not produce or properly use insulin, a hormone needed to convert sugar and starches into energy. Although the cause of diabetes is not completely understood, genetics, environmental factors, and viral causes have been partially identified.

There are two major types of diabetes: Type I and Type II. Type I diabetes is an autoimmune disease in which the body does not produce insulin and most often occurs in young adults and children. People with Type I diabetes must take daily insulin injections to stay alive.

Type II diabetes is a metabolic disorder resulting from the body's inability to make enough, or properly to use, insulin. Type II diabetes accounts for 90-95% of diabetes. In the United States, Type II diabetes is nearing epidemic proportions, principally due to an increased number of older Americans and a greater prevalence of obesity and a sedentary lifestyle.

Insulin, in simple terms, is the hormone that unlocks the cells of the body, allowing glucose to enter those cells and feed them. Since, in diabetics, glucose cannot enter the cells, the glucose builds up in the blood and the body's cells ‘starve’.

Diabetics having Type I diabetes typically are required to self-administer insulin using a syringe or a pin with needle and cartridge. Continuous subcutaneous insulin infusion via implanted pumps is also available. Insulin itself is typically obtained from pork pancreas or is made chemically identical to human insulin by recombinant DNA technology or by chemical modification of pork insulin. There are a variety of different insulins for rapid-, short-, intermediate-, and long-acting forms that may be used variously, separately or mixed in the same syringe.

It is highly recommended by the medical profession that insulin-using patients practice self-monitoring of blood glucose (SMBG). Based upon the level of glucose in the blood, individuals may make insulin dosage adjustments before injection. Adjustments are necessary since blood glucose levels vary day to day for a variety of reasons, e.g., exercise, stress, rates of food absorption, types of food, hormonal changes (pregnancy, puberty, etc.) and the like. Despite the importance of SMBG, several studies have found that the proportion of individuals who self-monitor at least once a day significantly declines with age. This decrease is likely due simply to the fact that the typical, most widely used, method of SMBG involves obtaining blood from a finger stick. Many patients consider obtaining blood to be significantly more painful than the self-administration of insulin.

Thus there is a strong and long felt need for less invasive methods of glucose measurement and monitoring. Some methods of glucose monitoring, use body fluids other than blood (e.g., sweat or saliva), subcutaneous tissue, or blood measured less invasively. Sweat and saliva are relatively easy to obtain, but their glucose concentration appears to lag in time significantly behind that of blood glucose levels.

Minimally invasive techniques known as ‘lancing’ employ micro-needles which lightly penetrate the skin. Needle-type sensors have been improved in accuracy, size, and stability and may be placed in the subcutaneous tissue or peripheral veins to monitor blood glucose with small instruments. See, “An Overview of Minimally Invasive Technologies”, Clin. Chem. 1992 September; 38(9):1596-1600.

CME telemetrix Inc. suggests a system called “GlucoNIR”. As the name implies, these systems use infrared spectroscopy in the near-IR spectrum. A beam of light is focused on a person's finger for about half a minute. By applying mathematical analysis to emerging light, concentration of various blood analytes including glucose may be determined.

InLight Solutions, previously associated with LifeScan and Johnson and Johnson presently has ties with researchers at the University of New Mexico, and has been working on another near-IR device. InLight's optics and software design is specific to distinguish the target molecule glucose, from similar molecules like water. Their devices are of three primary parts: a light source, an optical detector, and a spectrometer. The light shines into the skin and a small amount of that light will reflect back being analyzed by the spectrometer with subsequent measurement by the detector. Measuring the differences between the light that went into the skin with the light that the detector collects. Each molecule's response to the light can be distinguished by different vibration characteristics, thus the system can be programmed to analyze a particular molecule. This system is optimized to give an accurate quantitative measurement of glucose concentrations in the body.

Optiscan proposes another IR approach. Unlike Near-IR approaches, Optiscan measures glucose in the middle infrared or Mid-IR range. One big advantage of using this region of the IR spectrum is that there is minimal interference from other molecules like urea. Another is that blackbody radiation can be used, i.e., the device can actually use the human body's own inherent radiation, rather than apply an external radiation source. However, a big problem arises in that Mid-IR waves are largely absorbed by water, which is the major component of blood and interstitial fluid. Another is that because there is no temperature difference between water and glucose beneath the skin, they cannot be easily differentiated from each other. A rapid cooling device on the skin surface which detects glucose in a phase-shift using eight different wavelengths of IR radiation in the 9 to 10 micron range. In this way, Glucose can be detected very close to the skin surface to a depth of about 200 micrometers. Accuracy is reduced by individual variations in black body radiation, skin temperature, cooling speed and other factors. Sensys Medical Inc. has a near-infrared spectroscopy technology for it's non-invasive glucose monitor which addresses patients' skin directly. Interfacing layers of skin contribute to the variable reflection and refraction of light. Skin roughness also causes large specular reflectance. Changes in skin over time as well as skin temperature changes will contribute to difficulties.

Patented methods and devices relating to minimally invasive blood glucose measurement include the following.

U.S. Pat. No. 4,169,676 to Kaiser, shows methods of glucose measurement by putting a sensor directly against the skin or against the tongue. The procedure and device shown there uses a laser and determines the content of glucose in a specific living tissue sample by comparing the IR absorption of the measured material against the absorption of IR in a control solution by use of a reference prism.

Dahne et al., teach in U.S. Pat. No. 4,655,255, an apparatus for non-invasively measuring the level of glucose in a blood stream or tissues. These methods are photometric and use light in the infrared spectral region. These procedures use light in the 1.0 to 2.5 micron range. Dahne's device is jointly made up to two main sections, a light source and a detector section situated about a body part such as a finger. Infrared light is achieved by use of filters placed after a broadband source. The detector section is made up of a light-collecting integrating sphere or half-sphere leading to a means for detecting wavelengths in the near-infrared region. Dahne et al. goes to some lengths teaching away the use of IR light having wavelengths greater than about 2.5 microns since those wavelengths are strongly absorbed by water and have very little penetration capability into living tissues containing glucose.

Rosenthal et al., describes a non-invasive glucose monitoring device using near-IR light in their U.S. Pat. No. 5,028,787. Light is passed into the body in such a way that it passes through some blood-containing region. The so-transmitted or reflected light is then detected using an optical detector. The near-IR light sources are preferably infrared emitting diodes (IRED).

Harjumaa et al, teaches in U.S. Pat. No. 5,178,142, to use a stabilized near-IR radiation beam containing two alternating wavelengths in a device to determine a concentration of glucose or other constituents in a human or animal body. The amplitude of the varying alternating signal is detected and is said to represent glucose concentration or is taken to represent the difference in glucose concentration from a preset reference concentration.

U.S. Pats. No. 5,179,951 and 5,115,133, to Knudson, show application of IR light for measuring blood glucose levels directly in blood vessels in the tympanic membrane. Detected signals are amplified, decoded, and, using a microprocessor, provided to a display device. The IR detector includes “means for detecting the temperature of the volume in the ear between the detector and the ear's tympanic membrane.”

In U.S. Pat. No. 5,433,197, Stark describes a non-invasive glucose sensor. IR radiation is passed into the eye through the cornea and the aqueous humor, reflected from the iris or the lens surface, and then passed out through the aqueous humor and cornea. Reflected radiation is collected and detected by an IR sensor which measures the reflected energy in one or more specific wavelength bands. Comparison of reflected energy with source energy provides a measure of the spectral absorption by the eye components. Measured glucose concentration in the aqueous humor tracks that of the blood by a fairly short time. The infrared source is an LED with a refraction grating so that the light of a narrow wavelength band 10 to 20 nanometers wide passes through the exit slit. Use of IR spectrum below 1.4 microns and in the region between 1.5 and 1.8 microns is suggested.

U.S. Pat. No. 5,267,152, to Yang et al., shows a non-invasive method and device for measuring glucose concentration. Near-IR radiation, specifically with a wavelength of 1.3 microns to 1.8 microns from a semiconductor diode laser is used as an optical source. Light is transmitted down through the skin to the blood vessel where light interacts with various components of the blood and is then diffusively reflected by the blood back through the skin for measurement of the resulting spectrum.

Inventor Kuperschmidt presents a device in U.S. Pat. No. 5,398,681, which is said to be a pocket-type apparatus for measurement of blood glucose using polarization techniques. Glucose tends to rotate the polarization of light passing therethrough. Laser light is introduced into a finger or ear lobe and the phase difference between a reference signal and the measurement signal is measured and processed to formulate and calculate a blood glucose concentration which is then displayed.

U.S. Pat. No. 6,001,067 shows an implantable device suitable for glucose monitoring. It utilizes a membrane in contact with a thin electrolyte phase, which in turn is covered by an enzyme-containing membrane, e.g., glucose oxidase in a polymer system. Sensors are positioned in such a way that they measure the electro-chemical reaction of the glucose within the membranes. That information is then passed to desired sources.

Marchitto et al present yet another system for biological measurement which is minimally invasive as U.S. Pat. No. 6,387,059. In this teaching, pulsed light is used to form a microblister and draw interstitial fluid to the surface of the skin where it can be collected for chemical analysis. This technique, while using optical pulsed energy to draw a reaction from the tissue, does not directly measure the optical response in the tissue as a results of absorption or optical scatter.

A special class of non-invasive in vivo optical glucose measurement includes one based upon phenomena known as a ‘photoacoustic effect’ PA. The following patents relate primarily to systems employing photoacoustic effects.

U.S. Pat. No. 5,657,754 by inventor Rosencwaig primarily describes apparatus for the non-invasive analysis of non-homogeneous samples. The apparatus is suited for analyzing biological samples. An intensity modulated light beam is used to preferentially heat a selected constituent in the sample. Such periodic heating causes thermal waves in the test medium.

Inventor Chou of California teaches a method and apparatus for non-invasive measurement of blood glucose by photoacoustics in U.S. Pat. No. 5,941,821 published Aug. 24, 1999. Heating at the surface of a tissue causes surrounding air to also heat and produce a response signal which is measured or detected via a differential microphone. Further, optical energy is delivered to the tissue via a fiber optic coupling element. The same inventor further presents in a similar disclosure, U.S. Pat. No. 6,049,728 additional detail relating to these techniques.

Oraevsky and others teach special real time optoacoustic monitoring of changes in tissue properties via important optoacoustic imaging technique. U.S. Pat. No. 6,309,352 dated Oct. 10, 2001. These systems, while being directed to monitor tissues, are not suitable for blood analyte measurement but rather are aligned with the task of control during operational procedures occurring simultaneously with the measurement. Oraevsky additionally presents an interesting technique of analysis with regard to the spatial profile of an optically-induced acoustic transient. This recent disclosure is U.S. Pat. No. 6,405,069 dated Jun. 11, 2002. In some versions, the technique includes addressing tissue via the eye which has better access to certain components not readily available in techniques addressing tissue via dermal layers.

U.S. Pat. No. 6,466,806 of Oct. 15, 2002 presents very special technique characterized as resonant photoacoustic spectroscopy. This involves tuning optical pulses to cause a resonant acoustic wave. In accordance with a reference database, the parameters of the pulses which cause resonance suggests features of the material such as blood analytes concentration.

U.S. Pat. No. 6,526,298 by inventors Khalil et al, describes techniques with emphasis in temperature compensation. The skin sometimes is highly variable in temperature and tends to make difficulties in some measurement configurations. Accordingly, steps may be taken to reduce or compensate for temperature variances as taught by these researchers.

U.S. Pat. No. 6,567,688 includes a photoacoustic technique employing microwave energy to stimulate an acoustic response.

In contrast to good and useful inventions mentioned, each having certain features that are no less than remarkable, the instant invention is concerned with blood analyte measurement systems having quantum cascade lasers for the optical source. Inventions of the art are not used and cannot be used to realize the advantages and objectives of the inventions taught here following.

Documents Incorporated by Reference

The present inventors have taught related inventions in disclosures previously filed as patent applications. These disclosures include important elements and concepts which might be useful in a full understanding of the present inventions. Thus, those documents are incorporated in their entirety into this teaching by reference. These include U.S. patent applications having Ser. No. 10/656,376; filed Sep. 8, 2003; application having Ser. No. 10/695,358 filed on Oct. 28, 2003; and an application, serial number unknown or unassigned, entitled “Spatial Detectors for in-vivo Measurement of Bio Chemistry”, having docket number 262.1, filed on or about Sep. 8, 2003.

### SUMMARY OF THE INVENTIONS

Comes now, John Hefti, James Plante, and Joseph Page with inventions of non-invasive infrared biochemistry measurement systems. These inventions include devices and methods of measuring substances in human flesh by way of optical pulses generated in specialized optical sources. It is a primary function of these systems to provide measurement and monitoring tools for health maintenance.

In brief, these inventions may be characterized as measuring systems for detecting and quantifying substances found in tissues. They employ the principle of photoacoustic spectroscopy and are arranged about a special spectra characterized as middle infrared. Optical sources include those having at least one semiconductor laser coupled to a tissue test site via an optical path which permits transmission of optical energy into the tissue. Also, they include a detector system acoustically coupled to said tissue test site. Acoustic energy is generated by molecules which absorb optical energy at the target substance, or a component, or marker of the substance being measured. In particular, quantum cascade type semiconductor lasers are used to produces beams in several wavelengths. In best versions, optical sources include at least two discrete lasers, the lasers arranged to address the same tissue region.

High performance quantum cascade lasers may be operated to produce modulated output beams without complex optical domain modulators. Rather, since they are highly and directly responsive to applied currents, they can be operated in a direct modulation scheme whereby the output follows an applied electronic input signal.

As quantum cascade lasers tend to have highly divergent asymmetrical beams, some versions employ special optics to better couple these beams to tissue test sites associated with various substances under measure. For example, to couple the greatest amount of optical energy to test plane 30 microns below the skin surface, a beam shaping system is deployed as part of the optical path between the optical source and the tissue test site.

In addition to devices, these inventions include methods best described as methods of in-vivo substance measurement including steps: exciting a quantum cascade semiconductor laser to form an optical pulses of middle infrared optical radiation; causing optical radiation to fall incident upon human tissue; receiving an acoustic return signal which results from interaction between said optical radiation and substances under test; and determining from the received acoustic signal information about a substance being addressed.

### OBJECTIVES OF THESE INVENTIONS

It is a primary object of these inventions to provide systems to measure tissue chemistry and blood analytes in particular.

It is an object of these inventions to provide systems with highly specialized high performance optical sources.

A better understanding can be had with reference to detailed description of preferred embodiments and with reference to appended drawings. Embodiments presented are particular ways to realize the invention and are not inclusive of all ways possible. Therefore, there may exist embodiments that do not deviate from the spirit and scope of this disclosure as set forth by the claims, but do not appear here as specific examples. It will be appreciated that a great plurality of alternative versions are possible.

### BRIEF DESCRIPTION OF THE DRAWING FIGURES

These and other features, aspects, and advantages of the present invention will become better understood with regard to the following description, appended claims and drawings where:

FIG. 1 is block diagram to illustrate a system overview of important elements and their relation to each other;

FIG. 2 shows a version where several beams interact with a tissue test site;

FIG. 3 similarly shows a plurality of lasers arranged to interact with a tissue test site;

FIG. 4 illustrates special multi laser modules; and

FIG. 5 is a diagram of a spectrum of interest and important interaction features thereon.

### GLOSSARY OF SPECIAL TERMS

Throughout this disclosure, reference is made to some terms which may or may not be exactly defined in popular dictionaries as they are defined here. To provide a more precise disclosure, the following terms are presented with a view to clarity so that the true breadth and scope may be more readily appreciated. Although every attempt is made to be precise and thorough, it is a necessary condition that not all meanings associated with each term can be completely set forth. Accordingly, each term is intended to also include its common meaning which may be derived from general usage within the pertinent arts or by dictionary meaning. Where the presented definition is in conflict with a dictionary or arts definition, one must use the context of use and liberal discretion to arrive at an intended meaning. One will be well advised to error on the side of attaching broader meanings to terms used in order to fully appreciate the depth of the teaching and to understand all the intended variations.

Quantum Cascade Laser

Quantum cascade laser, QCL is a semiconductor laser characterized by a series of quantum well structures arranged in a repetitive structure which product coherent light in response to electron transitions between quantum wells.

Tissue Test Site

A tissue test site includes a volume region of tissue including the optical path and acoustic path in a photoacoustic spectroscopic system designed for tissue measurements.

Middle Infrared

Middle infrared is an optical portion of the electromagnetic spectrum characterized as those wavelengths between about 2-70 microns.

### PREFERRED EMBODIMENTS OF THESE INVENTIONS

In accordance with each of the preferred embodiments of these inventions, apparatus for and methods of non-invasive in vivo substance measurement are provided. It will be appreciated that each of these embodiments described include both an apparatus or method and that the apparatus or method of one preferred embodiment may be different than the apparatus or method of another embodiment.

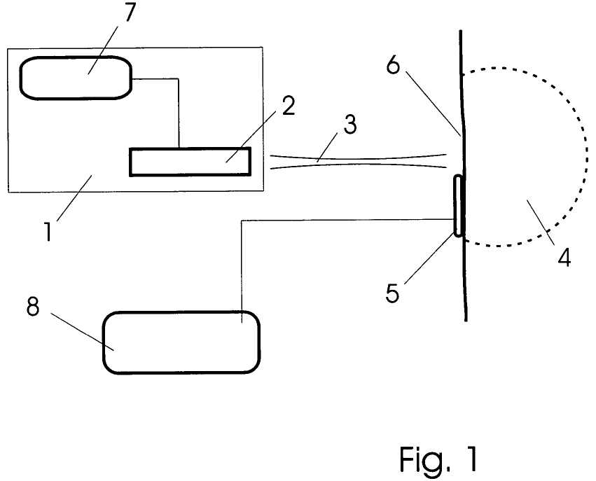

Most general versions of these inventions can be described as tissue substance measuring apparatus having an optical source with one or more lasers and a pressure transducer system each respectfully coupled to the same tissue test site. Coupling between the laser(s) and the tissue is by way of an optical path which permits transmission of optical energy characterized as middle infrared, Mid-IR, from the optical source into said tissue. Coupling between the pressure transducer system and the tissue is made via an acoustic path. In some preferred versions, a pressure transducer system is in direct contact with tissue and the acoustic path wholly within the tissues test site. As such, these devices are suitable for receiving acoustic energy which is generated as a result of absorption of the optical energy by a substance of interest, or in some cases, a component of a substance of interest or other marker related to a substance of interest. FIG. 1 illustrates a block diagram with these major elements shown in relation to one another. In particular, an optical source 1 contains lasers or a laser 2 which are coupled via an optical path 3 to a tissue test site 4. In addition, an acoustic or pressure detector system 5 which may be arranged to lie directly on a tissue surface 6. Electronic support includes a laser driver 7 comprised of pulse generation and timing systems appropriate for driving quantum cascade type lasers. Also, electronic support may include signal processing, data acquisition, data transmission means 8 coupled to the pressure transducer system.

In preferred versions, highly specialized optical sources are used. Optical sources of these devices and methods are comprised of at least one semiconductor laser. A Mid-IR semiconductor laser which is tunable and very small in size, yet quite powerful is known as a ‘quantum cascade laser’ QCL. Accordingly, as these properties are well aligned with some of the important objectives of these inventions, QCLs form the basis of most preferred optical sources.

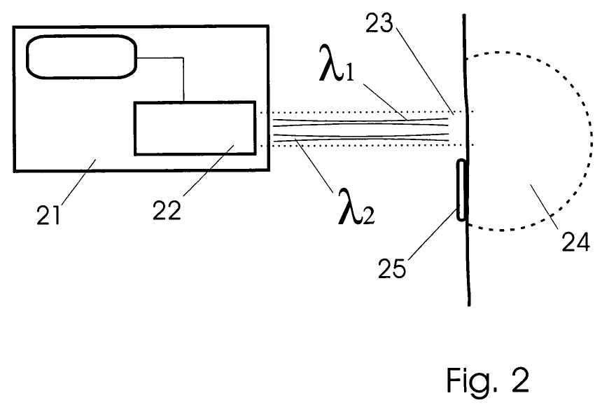

In optical sources having more than one laser, each laser may be provided to operate on a different wavelength. These wavelengths are tuned to cooperate with the natural absorption spectra of a target substance. For example, in blood glucose monitoring systems lasers might be tuned to align with features and artifacts of the glucose absorption spectrum. FIG. 2 illustrates in block diagram, an optical source having two lasers. Optical source 21 is an integrated module of electronic support and group of semiconductor lasers 22. These lasers might be arranged to produce two independent Mid-IR optical beams at wavelengths λ₁ and λ₂. In some versions, these beams might not precisely occupy the same optical path, but be contained within a limited optical space 23 which will be considered the ‘optical path’. Both beams address the same tissue sample site 24 while actually interacting with distinct volumes of tissue closely spaced. In this fashion, tissue excited by both beams will produce an acoustic signal readily detected by a detection transducer 25.

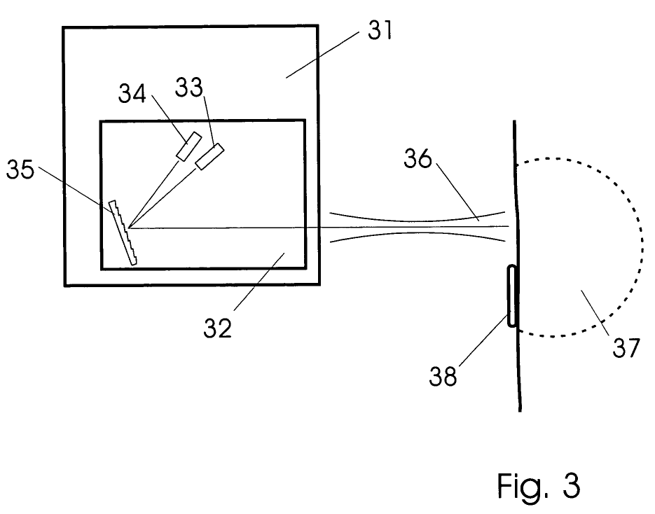

Sometimes, it is preferred that the beams from each laser of an optical source occupy precisely the same volume and the plurality of beams interact with exactly the same tissue space. Accordingly, two Mid-IR beams from QCLs might be combined into a single space with a beam combiner element such as a polarization beam combiner (sometimes more commonly known as a “beam splitter” for its use in the reverse direction.) Use of polarization beam combiners present many problems with the particular task at hand. A first problem is that the output of QCLs is not of such a ‘clean’ polarization profile unlike the ubiquitous gas laser with Brewster windows and a very high degree of linear polarization. Further, where three or more lasers are used, orthogonal polarizations are no longer available. For this reason, it is sometimes an advantage to arrange a plurality of lasers in conjunction with a beam combining element such as a diffraction grating to cause beams of different wavelengths to become colinear and to share identical space. FIG. 3 illustrates.

An optical source 31 is comprised of a special laser module 32 having two lasers 33 and 34 therein. The lasers are displaced and slightly rotated in angle. Their beams fall incident upon a reflective diffraction grating 35 where the beams of different wavelength are combined into a common beam which propagates along optical path 36 towards the tissue test site 37. The angle of rotation between the two lasers will be dictated by the grating or prism element which will diffract each wavelength at a different angle as is well known in optical sciences. Each beam, operated at different times, produces a different acoustic response in the tissue which is detected at the acoustic detection system 38. Beams may also be similarly combined in a prism type refractive beam combiner element.

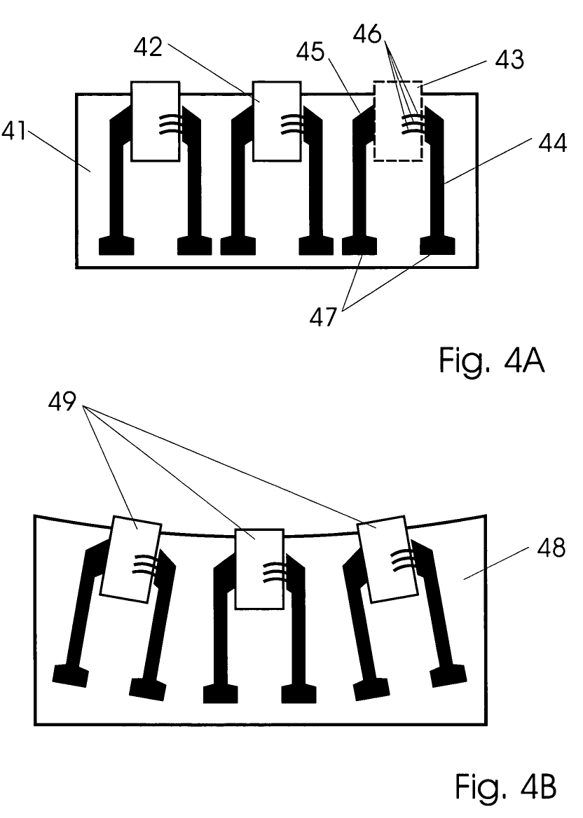

In some special systems, an optical source is configured with a plurality of lasers integrated together on a single carrier element. This is primarily done to further reduce the size of the entire apparatus such that it may more readily be made portable and user friendly. To effect this, QCL die are formed as crystal devices on the order of one or two millimeters in length each. These dice can be mounted into receiving stations on a carrier which has electrical leads appropriate for high current, high speed operation. With reference to FIGS. 4A and 4B, a common carrier 41, 48 might be arranged to accommodate several lasers 42 in a very limited space. For example, three QCLs 49 might be mounted into a carrier substrate of just two square centimeters or less. The carrier substrate might have thereon, three pads 43 appropriate for bonding the lasers to form electrical contact to either of the traces in a trace pair 44-45. These electrical traces may be formed in common lithographic processes. A second electrical contact may be made via a special wire bond system. Because QCLs are operated in very high current modes, common wire bonds are sometimes not of sufficient gauge to deliver sufficient current density to a laser. Therefore, a plurality of wire bonds 46 might couple one of the traces to a top side electrical contact of the QCL. These special arrangements of current carrying conductors are part of an advanced direct modulation means. The conductors are arranged to support fast switching and high currents which are preferably used in systems demanding high energy short pulses. QCLs used in other applications would more typically be found without special electrical couplings. FIG. 4B shows the special case where the lasers have an angular offset such that beams of different wavelengths might be combined into a single beam as desired in most versions. Although the diagram has been prepared with three lasers, this was done for clarity. In best versions, a carrier substrate has between two and ten individual laser elements.

In cases where the tissue penetration is difficult and great power is needed to achieve depths not possible with the output of a single laser, several lasers each lasing on the same wavelength may be combined to form one beam of considerably high power. In this way, tissue not addressable by one laser can be reached by lasers ganged together. A special grating can be used to combine two lasers of a single wavelength as diffraction on the +1 and −1 orders can share output space, the lasers being at different locations. At present, QCLs which are high current devices generally will not exceed 1 watt in power output. Alternatives are also possible. Even when very short pulses are used, the total amount of energy is limited by the maximum current the device can withstand. Thus, when two laser are run in parallel, the total power in a single beam may be doubled. In this way, one version of these inventions includes that where the optical source includes more than one laser operating on the same wavelength at the same time.

Further, it is possible to have a single laser operate in two wavelengths. This requires a special stack of quantum wells where the well thicknesses are designed with a view to creating two distinct electron transitions between two energy levels, a distinct transition is associated with each lasing wavelength.

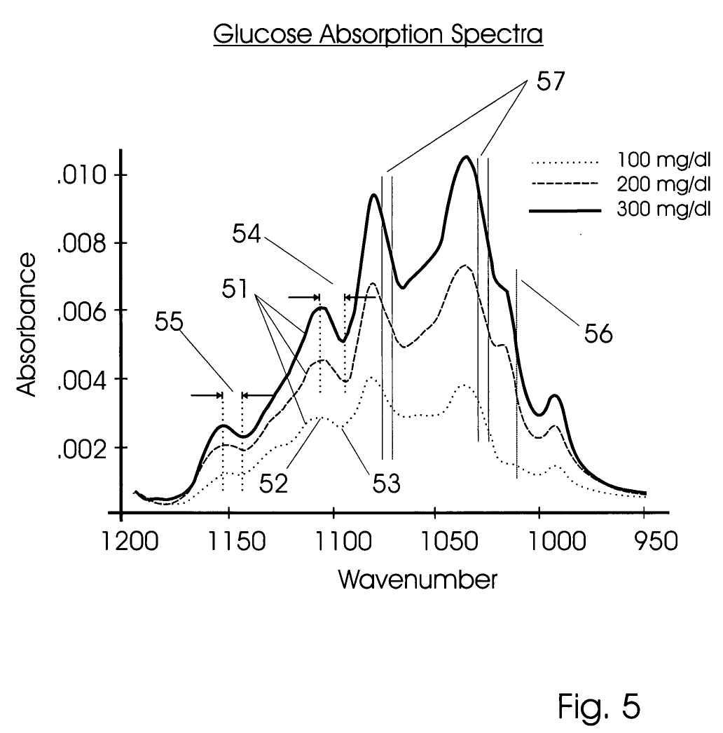

Still further, in a special case, one might drive a well designed laser with various voltages to alter its lasing wavelength. This can generally only be done to accomplish very limited differences in lasing wavelength. The technique is not practical for large wavelength differences. Thus, it is only applied in special arrangements where small wavelength changes result in a large effect on a cooperating system. An example of such system is illustrated in FIG. 5. An optical source is designed with lasers at wavelengths to interact with glucose spectra 51 at points on the spectra where the slope of the absorption line is substantially non-zero or preferably maximized. At local maxima and minima, or ‘peaks’ 52 and ‘valleys’ 53, the slope of the absorption curve is zero. Useful systems might be made where lasers are tuned to these corresponding wavelengths. In alternative systems it is desirable to operate with very small wavelength changes. These might be produced as a result of altering the voltage applied to a QCL to cause it to lase at two different wavelengths (centers). Since peaks are largely separated 54, 55 (in wavelength) from valleys, operation at those points might be impossible where a voltage controlled wavelength tuned QCL is employed. Wavelength bands 57 of largest 56 or non-zero slope provide workable systems in view of these special lasers. A laser may be tuned to emit light on a center wavelength on either side of a slope inflection point, a point where the slope has a transition from increasing slope to decreasing slope, or decreasing slope to increasing slope, to cause a maximum absorption response difference between the two slight wavelength changes. Some preferred version include where the two center wavelengths are symmetrically positioned about an inflection point. In this type of system, lasers should be tuned to wavelengths where the slope of the absorption curve is non-zero; i.e. away from peaks and valleys. In alternative systems where several lasers are available without wavelength tuning restrictions, absorption curve maxima and minima remain good points to operate.

While use of QCL in biochemistry detection systems has many advantages clearly indicated herein, their use also come with some difficulties. For example, the output beam of a QCL is highly asymmetric in cross section. Further, its output is highly divergent; and its divergence in a first plane is different than its divergence in an orthogonal plane. Since optical output from QCLs are generally in the Mid-IR, beam shaping optics must be made of compatible materials or configurations such that those optics cooperate with the beam. For example, common glass used to form lenses cannot be used with QCL outputs as it is highly opaque at long wavelengths. Reflective optics are preferred. Where reflective optics are not available, then special materials such as GaAs might be used to form refractive optics. Thus, to properly couple a QCL to tissues in measurement systems, special considerations must be taken. More particularly, when a QCL is employed in a photoacoustic glucose measurement system, additional factors become important. These include pulse energy, pulse width, spot size, divergence-convergence, among others.

It is essential that the proper pulse energy be used. If too great of pulse energy is applied, tissue damage will result. If too low of pulse energy is used, the beam will not penetrate deep enough to interact with glucose which can only be found deep (greater than about 10 microns) within the tissue.

While common lasers are regularly configured to produce beams of terawatts in average power as well as beams of nanowatts, these power levels are not appropriate for the task at hand. Rather, the beams appropriate for probing tissue test sites in photoacoustic Mid-IR are more likely to fall in the range of between about 10 watts and 50 milliwatts. In this range, the total energy is not sufficient to cause ablation of appreciable level, nor is it likely to be completely absorbed at the tissue surface. More precisely, beams having between 1 watt and 200 milliwatts average power are preferred.

Too small of a beam cross section or spot-size results in a prohibitively high energy density and might have the undesirable result of causing tissue burns. Conversely, too large of a beam spot-size energy will completely be absorbed at the tissue surface and no energy will get below 10 microns where it can properly interact with a desired analyte. Thus preferred spot sizes are at least 30 microns and no greater than 2 mm at the beam waist. While it is not a requirement that a beam waist be in the plane of a tissue surface, some preferred versions are arranged such that a laser beam comes to a waist or focus at the tissue surface whereby energy density is maximized at the same time tissue cross section is minimized; optimal conditions for increasing the penetration of light into deeper levels of tissue being tested.

The intrinsic characteristics of a QCL suggest that the lasers cross section be rectangular and asymmetric. That is, its exit aperture is necessarily a rectangle. This has the undesirable effect of producing a beam with a different divergence in two orthogonal directions. Accordingly, special cylindrical optics can be placed in an optical path to manipulate the output beam into a form which better promotes penetration into a tissue. For example, where a tissue target plane is at a prescribed depth, the beam may be manipulated such that the ‘spot’ is of similar size in two dimensions as nearly as practical. In view of differing divergence, this only can occur at one plane; thus there lies advantage in causing it to occur in the plane of greatest interest a plane at a predetermined test depth. To better effect this, an anisotropic special mirror (might be included in the optical path. A mirror may be used as Mid-IR wavelengths demand special materials for transmissive optics.

Many laser based systems depend upon beams of light having an intensity variable in time. To effect this, a modulation scheme is engaged. Most typically, modulation occurs in the optical domain immediately after the output of the laser in the optical train. For example, a light shutter such as a liquid crystal modulator may be used. More commonly found might be the electro-optic modulator which manipulates polarization or phase to modulation the beam. While use of these systems is certainly possible in photoacoustic spectroscopy, a novel and simplified modulation scheme is preferred in the present inventions.

Because the nature of photoacoustic spectroscopy as it relates to QCLs permits short pulses on timing schemes which cooperate with direct stimulation by electrical currents of the laser, a direct modulation scheme is preferred. Direct modulation simplifies the entire apparatus as it reduces the optical complexity.

For purposes of these inventions, direct modulation is effected by driving a direct current through a quantum well structure is short pulses or short bursts of pulses. A short pulse or ‘delta function’ pulse means one of which is less than 1/10 of a timing cycle. In some cases, a short pulse will be 1/10,000^{th} of a timing cycle. These currents may be arranged in view of the lasers' lasing threshold and further in view of preferred timing and duty cycles which produce best acoustic return signals. In one preferred version, a series of short pulses separated by between one and ten milliseconds causes a desirable heating response in a tissue test site to produce a readily detectable acoustic return wave.

Direct modulation cannot work with lasers such as gas, dye, or chemical lasers because their turn-on and turn off cycles, and pumping mechanisms don't cooperating with the timing required in photoacoustic spectrographic systems. A QCLs lasing mechanism is particularly responsive to currents applied there through and thus they support direct modulation.

Since QCLs are adversely affected by high duty cycles—they are high current devices they and will melt if run too long with such high currents—it is advantageous to operate a QCL with short pulses and long resting times. QCLs used in some digital communications systems might be tasked with a very long ‘ON’ period and thus those QCLs might be better configured for continuous wave operation. In contrast, the nature of the photoacoustic principle actually prefers very short or ‘delta function’ type pulses. In agreement, special QCLs which can have very high instantaneous outputs but very low duty cycles are preferred for these inventions. This can be accomplished in laser configurations having no mirrors for example. Mirrors will support continuous wave lasing but severely limit heat transfer. Without mirrors and with sufficiently high currents and long resting times, a QCL can produce very high peak power pulses. Thus, in photoacoustic systems based upon QCL lasers, it is preferred that the duty cycle be no more than 1:5. Ideally, a duty cycle of 1:100 or even 1:1000 or greater might be the best operation for preferred versions.

Ideally, 500 microsecond pulses permit a QCL to run at a 1 KHz repetition rate without excessive heating problems. Of course, the pulse duration can be greatly shortened to 100 microseconds or less to get the desired result of an even lower duty cycle. A repetition rate of the order between 100 hertz and 1 kilohertz is useful as it cooperates well with the acoustic range of easily configurable acoustic detectors and media such as acoustic cells and conduits. Repetition rates on the order of one second due not cooperate well with the tasks at hand because sound travels too fast in tissue to permit any standing wave activity which can be used to enhance a detection system response. Conversely, a repetition rate of 100 kilohertz or more exceeds the speed at which heating of a sample provides a quality acoustic wave and those rates should be avoided. Thus a repetition rate between about 10 hertz and 10 kilohertz is preferred.

In some special versions of these inventions, the time between successive pulses is a critical factor in spatial resolution schema. Where multi-element directional detectors are used, the modulators timing and switching mechanisms maybe arranged to cooperate with the physical distribution of detector elements. In this way, another preferred version may be realized. By using constructive and destructive superposition, successive pulses are added and subtracted to one another in a phase manipulation arrangement. This allows a detector to ‘look’ to various regions of the tissue in a steered detector array. The pulse rate of the modulator must be configured to agree with the spatial distribution of the detector elements.

While best versions are arranged with very short pulse lengths, it is prohibitively difficult to deliver large currents in very short time periods through a device of only several square millimeters. Thus, a ‘delta function’ pulse is herein meant to be those which are between about 10 ns and 1 ms. Pulses which are greater than 1 ms in length would tend to defeat the preferred periodic heating necessary to properly produce acoustic vibrations. So, a QCL driven in accordance with these pulse width ranges is particularly arranged to cooperate with a Mid-IR photoacoustic detection scheme.

While some interesting versions include those having an acoustic resonance cell, preferred alternatives may include those whereby direct coupling between a transducer and tissue is provided. A pressure transducer is coupled to the tissue test site at the surface of the tissue, the skin. The pressure transducer makes intimate and direct contact with a tissue surface and acoustic energy is passed into the transducer from the tissue.

To make a more efficient junction for passing acoustic energy, a tissue test site may be coupled via a fluid operable for transmitting an acoustic wave there through. The fluid tends to occupy the irregular spaces of the tissue surface which otherwise degrade the transmission.

Because space is at an extreme premium in small wearable devices, one arrangement includes a system abbreviated to exclude non essential components. For example, it is not necessary that these apparatus include a display means to present information to a user. Where the system is performing a long term recording of glucose changes for use by a physician, a system without direct feedback to the patient, it may be preferable to arrange a data storage means which merely collects and records a series of signals. These signals could later be passed to a processing system, for example the personal computer of a physician office. The data could be analyzed in accordance with a treatment plan and the user would be passed new instructions by the doctor. Alternatively, its outputs could be arranged to drive an insulin pump automatically where no indication of the glucose levels are ever known by the user. In such cases, a data transmission means is included to pass stored data to an independent system for post processing or archiving.

These inventions also include methods. More precisely, methods of in-vivo substance measurement having steps: exciting a quantum cascade semiconductor laser to form an optical pulse set of middle infrared optical radiation; causing the optical pulse set to fall incident upon tissue; receiving an acoustic return signal; and determining from the received acoustic signal information about a substance being addressed. Further, the laser may be excited in a direct modulation process whereby a pulse set is formed by applying current pulses through the QCL to effect periodic lasing. This is meant to include the case where short current pulses are used and such pulses are characterized as ‘delta function’ pulses. A finite set of pulses may include between 2 and 2000 to form a ‘pulse set’ of finite length. The time between individual pulses can be arranged cooperate with a spatial arrangement of a pressure transducer system in view of the speed of acoustic waves in tissues being measured.

The illumination step can include such action as illuminating interstitial fluid just below the epidermis as it is not necessary to illuminate blood to learn glucose levels. Interstitial fluid has a glucose concentration which lags blood glucose by only a few or few 10's of minutes and blood glucose concentration might be inferred from an interstitial fluid measurement. This is important because blood is sometimes too deep in the tissue to reach with Mid-IR light. Interstitial fluid might be more accessible or made more assessable by drawing to the skin surface.

These methods further include providing optical energy of a sufficient quantity such that a substantial portion of the energy will penetrate into tissue to a depth between 20-100 microns below the tissue surface.

One will now fully appreciate how tissue chemistry may be measured and detected in vivo by way of special systems including unique optical sources. Although the present inventions have been described in considerable detail with clear and concise language and with reference to certain preferred versions thereof including the best mode anticipated by the inventor, other versions are possible. Therefore, the spirit and scope of the invention should not be limited by the description of the preferred versions contained therein, but rather by the claims appended hereto.

1 Tissue substance measuring apparatus comprising:
an optical source comprising at least one semiconductor laser, the optical source being coupled to a tissue test site via an optical path which permits transmission of optical energy characterized as middle infrared from said optical source into said tissue; and a pressure transducer system acoustically coupled to said tissue test site.

2 Tissue substance measuring apparatus of claim 1, wherein said pressure transducer is arranged to receive acoustic energy generated as a result of absorption of the optical energy at the substance, or a component or marker of the substance, being measured.

3 Tissue substance measuring apparatus of claim 2, said at least one semiconductor laser includes a laser of the type known as a quantum cascade laser comprised of quantum well structures.

4 Tissue substance measuring apparatus of claim 3, said optical source comprises at least two discrete lasers, said lasers being arranged to address or couple with a common region of tissue.

5 Tissue substance measuring apparatus of claim 4, said at least two discrete lasers each operate on a different wavelength.

6 Tissue substance measuring apparatus of claim 5, the at least two discrete lasers are coupled via an optical multiplexer to form a beam which addresses the same tissue, the lasers being operable independently in time.

7 Tissue substance measuring apparatus of claim 6, the multiplexer is an optical beam multiplexing device known as a beam combiner.

8 Tissue substance measuring apparatus of claim 6, the multiplexer is an optical beam multiplexing device known as a grating or prism type beam combiner.

9 Tissue substance measuring apparatus of claim 6, at least two discrete lasers are ranged to address substantially the same space or at least space having similar characteristics.

10 Tissue substance measuring apparatus of claim 1, said pressure transducer is coupled to the tissue test site via a coupling whereby the pressure transducer makes intimate and direct contact with a tissue surface.

11 Tissue substance measuring apparatus of claim 10, said pressure transducer is coupled to said tissue test site via a coupling comprised of a fluid operable for transmitting an acoustic wave therethrough.

12 Tissue substance measuring apparatus of claim 1, said optical source is further comprised of a modulation system electronically connected to said at least one semiconductor laser, the modulation system provides electronic pulses directly through said quantum cascade laser structure to directly modulate output beams.

13 Tissue substance measuring apparatus of claim 12, said modulation system is further comprised of a timing system and switch operable for delivering pulses characterized as delta function pulses.

14 Tissue substance measuring apparatus of claim 13, said pulses are formed into sets of pulses, or a pulse stream, of finite length, said sets of pulses being characterized as having a duty cycle less than ¼.

15 Tissue substance measuring apparatus of claim 13, said timing system and switch operable for delivering pulse trains which cooperate with a pressure transducer system having a spatial distribution.

16 Tissue substance measuring apparatus of claim 1, wherein the optical source generates optical energy in a plurality of wavelength bands each having a center wavelength, the center wavelengths being arranged to correspond to portions of a glucose absorption spectrum where the slope is substantially non-zero.

17 Tissue substance measuring apparatus of claim 16, wherein the optical source generates optical energy in at least two wavelength bands each having a center wavelength, the center wavelengths being arranged to correspond to portions of a glucose absorption spectrum on either side of a point where the slope has a transition from increasing slope to decreasing slope, or decreasing slope to increasing slope.

18 Apparatus of claim 17, said center wavelengths are arranged to lie symmetrically about the same inflection point.

19 Tissue substance measuring apparatus of claim 1, further comprising a data storage means coupled with said pressure transducer system whereby amplitude data may be recorded and stored by the apparatus.

20 Tissue substance measuring apparatus of claim 19, the apparatus further comprises a data transmission means arranged to pass stored data to an independent system for post processing and archiving.

21 Tissue substance measuring apparatus of claim 1, said optical source comprises at least two semiconductor lasers of the same wavelength.

22 Methods of in-vivo substance measurement, comprising the steps:
a) exciting a quantum cascade semiconductor laser to form an optical pulse set of middle infrared optical radiation;
b) causing said optical pulse to become incident upon human tissue being measured;
c) receiving an acoustic return signal which results from interaction between said optical pulse set and substances from which said tissue is comprised; and
d) determining from the received acoustic signal information about a substance being addressed.

23 Methods of claim 22, said ‘exciting’ step is a direct modulation step whereby by current pulses are driven through said semiconductor laser to effect periodic lasing.

24 Methods of claim 23, said current pulses are characterized as delta function pulses at a rate of between 10 hz to 10 khz.

25 Methods of 23, said current pulses include pulses arranged in a set of finite number.

26 Methods of claim 25, the period of pulses is arranged cooperate with a spatial arrangement of a pressure transducer system in view of the speed of acoustic waves in tissues being measured.

27 Methods of claim of 22, the ‘causing the optical pulse’ step is further defined as illuminating interstitial fluid just below an epidermis layer in a living human.

28 Methods of claim 27, said ‘causing pulse to propagate’ step is further defined as providing optical energy of a sufficient quantity such that a substantial portion will penetrate into tissue to a depth between 20-100 microns below the tissue surface.
